(19) United States
(12) Patent Application Publication
(10) Pub. No.: US 2024/0307704 A1
(43) Pub. Date: Sep. 19, 2024

(54) PHOTOTHERAPEUTIC ILLUMINATION DEVICES AND LIGHT GUIDE ASSEMBLIES WITH EMISSION-DIRECTING STRUCTURES

(71) Applicant: KNOW Bio, LLC, Morrisville, NC (US)

(72) Inventors: David T. Emerson, Durham, NC (US); P. Joseph DeSena, Jr., Raleigh, NC (US); Steve Reich, Durham, NC (US); Antony van de Ven, Nong Kae (TH); James Michael Lay, Apex, NC (US); Thomas Matthew Womble, Wake Forest, NC (US)

(21) Appl. No.: 18/184,129

(22) Filed: Mar. 15, 2023

(51) Int. Cl. A61N 5/06

(52) U.S. Cl. CPC A61N 5/0603; A61N 2005/0606; A61N 2005/0666

(57) ABSTRACT

Devices for impinging light on tissue to induce one or more biological effects and more particularly phototherapeutic illumination devices and light guide assemblies with emission-directing structures are disclosed. Emission-directing structures include arrangements of light guides and light guide positioners that may be provided with titled arrangements for directing light emissions toward target tissue. An exemplary light guide may define an optical axis therethrough, and an exemplary light guide positioner may include one or more features that are tilted with respect to the optical axis. Certain tilted features may relate to offset incisor tabs and/or angled biting surfaces that engage within the oral cavity for repeatedly orienting the optical axis in a target direction.

### FIELD OF THE DISCLOSURE

The present disclosure relates generally to devices for impinging light on tissue to induce one or more biological effects and more particularly to phototherapeutic illumination devices and light guide assemblies with emission-directing structures.

### BACKGROUND

Disease-causing pathogens typically invade tissues of the human body via mucosal surfaces within body cavities, such as mucous membranes or mucosae of the respiratory tract. A number of respiratory diseases and infections, including viral and bacterial, can be attributed to such disease-causing pathogens. Examples include Orthomyxoviridae (e.g., influenza), common colds, coronaviridae (e.g., coronavirus), picornavirus infections, tuberculosis, pneumonia, bronchitis, and sinusitis. Most respiratory tract infections begin when a subject is exposed to pathogen particles, which enter the body through the mouth and nose. For viral infections, cells at the site of infection must be accessible, susceptible, and permissive for the virus, and local host anti-viral defense systems must be absent or initially ineffective. Conventional treatments for infections may involve systemic administration of antimicrobials, such as antibiotics for bacterial infections, that can sometimes lead to drug resistance and gastro-intestinal distress. Other conventional treatment protocols may involve managing and enduring symptoms while waiting for infections to clear, particularly in the case of viral infections.

Upper respiratory tract infections, including the common cold, influenza, and those resulting from exposure to coronaviridae are widely prevalent infections that continually impact the worldwide population. In some instances, upper respiratory tract infections can progress to cause serious and sometimes fatal diseases that develop in the lower respiratory tract or elsewhere in the body. The art continues to seek improved treatment options for respiratory tract conditions capable of overcoming challenges associated with conventional treatment options.

### SUMMARY

The present disclosure relates generally to devices for impinging light on tissue to induce one or more biological effects and more particularly to phototherapeutic illumination devices and light guide assemblies with emission-directing structures. Emission-directing structures include arrangements of light guides and light guide positioners that may be provided with titled arrangements for directing light emissions toward target tissue. An exemplary light guide may define an optical axis therethrough, and an exemplary light guide positioner may include one or more features that are tilted with respect to the optical axis. Certain tilted features may relate to offset incisor tabs and/or angled biting surfaces that engage within the oral cavity for repeatedly orienting the optical axis in a target direction.

In one aspect, a light guide assembly comprises: a light guide configured to be optically coupled to at least one light source, the light guide comprising a base end that is proximate the at least one light source, a distal end, and a center region that is between the base end and the distal end; and a light guide positioner arranged about a periphery of the center region, the light guide positioner comprising a first incisor tab and a second incisor tab that are positioned on opposing sides of the center region, the first incisor tab being positioned closer to the base end of the light guide than the second incisor tab. In certain embodiments, the first incisor tab is arranged to engage with a back surface of an upper incisor and the second incisor tab is arranged to engage with a back surface of a lower incisor to position a portion of the light guide within an oral cavity of a patient. In certain embodiments, the light guide defines an optical axis in a direction from the base end to the distal end; the light guide positioner comprises an upper biting surface and a lower biting surface; and the lower biting surface is angled toward the optical axis such that at least a portion of the upper biting surface is closer to parallel with the optical axis than the lower biting surface. In certain embodiments, the light guide defines an optical axis in a direction from the base end to the distal end, and a tilt angle is formed between a first line perpendicular to the optical axis and a second line that intersects the first incisor tab and the second incisor tab, the tilt angle being in a range from 5 to 22 degrees, or in a range from 5 to 20 degrees. The light guide assembly may further comprise a tongue depressor that extends from the distal end of the light guide.

In another aspect, an illumination device comprises: at least one light source; driver circuitry configured to drive the at least one light source; a light guide optically coupled to the at least one light source, the light guide comprising a base end that is proximate the at least one light source, a distal end, and a center region that is between the base end and the distal end; and a light guide positioner arranged about a periphery of the center region, the light guide positioner comprising a first incisor tab and a second incisor tab that are positioned on opposing sides of the center region, the first incisor tab being positioned closer to the base end of the light guide than the second incisor tab. In certain embodiments, the first incisor tab is arranged to engage with a back surface of an upper incisor and the second incisor tab is arranged to engage with a back surface of a lower incisor to position a portion of the light guide within an oral cavity of a patient. In certain embodiments, the light guide defines an optical axis in a direction from the base end to the distal end; the light guide positioner comprises an upper biting surface and a lower biting surface; and the lower biting surface is angled toward the optical axis such that at least a portion of the upper biting surface is closer to parallel with the optical axis than the lower biting surface. In certain embodiments, the light guide defines an optical axis in a direction from the base end to the distal end, and a tilt angle is formed between a first line perpendicular to the optical axis and a second line that intersects the first incisor tab and the second incisor tab, the tilt angle being in a range from 5 to 22 degrees. In certain embodiments, the tilt angle is 6 degrees with a tolerance of plus or minus 1 degree. The illumination device may further comprise a tongue depressor that extends from the distal end of the light guide. The illumination device may further comprise a lens positioned between the at least one light source and the light guide, wherein an apex of the lens is positioned within the light guide.

In another aspect, an illumination device comprises: at least one light source; driver circuitry configured to drive the at least one light source; a light guide optically coupled to the at least one light source, the light guide comprising a base end that is proximate the at least one light source, a distal end, and a center region that is between the base end and the distal end, the light guide forming an optical axis in a direction from the base end to the distal end; and a light guide positioner arranged about a periphery of the center region, the light guide positioner comprising an upper biting surface, a lower biting surface, and an inflection point defined between the upper biting surface and the lower biting surface, the inflection point being offset from the optical axis. The illumination device may further comprise a tongue depressor that extends from the distal end of the light guide. In certain embodiments, the optical axis is positioned between the inflection point of the light guide positioner and the tongue depressor. In certain embodiments, the light guide positioner comprises a first incisor tab proximate the upper biting surface and a second incisor tab proximate the lower biting surface, and the first incisor tab is positioned closer to the base end of the light guide than the second incisor tab. In certain embodiments, a tilt angle is formed between a first line perpendicular to the optical axis and a second line that intersects the first incisor tab and the second incisor tab, the tilt angle being in a range from 5 to 22 degrees, or in a range from 5 to 20 degrees. In certain embodiments, the lower biting surface is angled toward the optical axis such that at least a planar portion of the upper biting surface is closer to parallel with the optical axis than the lower biting surface.

In another aspect, any of the foregoing aspects individually or together, and/or various separate aspects and features as described herein, may be combined for additional advantage. Any of the various features and elements as disclosed herein may be combined with one or more other disclosed features and elements unless indicated to the contrary herein.

Those skilled in the art will appreciate the scope of the present disclosure and realize additional aspects thereof after reading the following detailed description of the preferred embodiments in association with the accompanying drawing figures.

### BRIEF DESCRIPTION OF THE DRAWING FIGURES

The accompanying drawing figures incorporated in and forming a part of this specification illustrate several aspects of the disclosure, and together with the description serve to explain the principles of the disclosure.

FIG. 1 is a cross-section of an illumination device with emission-directing structures according to the present disclosure.

FIG. 2 is a partial cross-section illustrating placement of the illumination device of FIG. 1 partially within an oral cavity of patient.

FIG. 3 is a front perspective view of the illumination device of FIG. 1.

FIG. 4 is a side view of the illumination device of FIG. 3.

FIG. 5A is a side view of a portion of the illumination device of FIG. 3 that illustrates a light guide assembly with the light guide positioner arranged about the light guide.

FIG. 5B is a table listing parameters for a study of various jaw anatomies and simulated tilt angle α values according to aspects of the present disclosure.

FIG. 6A is a perspective view of the light guide of FIG. 5A illustrating a base end, a distal end, and a central region of the light guide.

FIG. 6B is a perspective view of the light guide positioner of FIG. 5A.

FIG. 6C illustrates a perspective view of the fully assembled light guide assembly that includes both the light guide of FIG. 6A and the light guide positioner of FIG. 6B.

FIG. 7A is an illustration of an image with an illumination device positioned in a patient's oral cavity where the illumination device is not arranged with a tilted arrangement between the light guide positioner and the light guide.

FIG. 7B is an illustration of an image with the illumination device according to FIGS. 1-6C that includes the above-described titled arrangement in the same patient's mouth as FIG. 7A.

### DETAILED DESCRIPTION

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element such as a layer, region, or substrate is referred to as being “on” or extending “onto” another element, it can be directly on or extend directly onto the other element or intervening elements may also be present. In contrast, when an element is referred to as being “directly on” or extending “directly onto” another element, there are no intervening elements present. Likewise, it will be understood that when an element such as a layer, region, or substrate is referred to as being “over” or extending “over” another element, it can be directly over or extend directly over the other element or intervening elements may also be present. In contrast, when an element is referred to as being “directly over” or extending “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

Relative terms such as “below” or “above” or “upper” or “lower” or “horizontal” or “vertical” may be used herein to describe a relationship of one element, layer, or region to another element, layer, or region as illustrated in the Figures. It will be understood that these terms and those discussed above are intended to encompass different orientations of the device in addition to the orientation depicted in the Figures.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this disclosure belongs. It will be further understood that terms used herein should be interpreted as having a meaning that is consistent with their meaning in the context of this specification and the relevant art and will not be interpreted in an idealized or overly formal sense unless expressly so defined herein.

Embodiments are described herein with reference to schematic illustrations of embodiments of the disclosure. As such, the actual dimensions of the layers and elements can be different, and variations from the shapes of the illustrations as a result, for example, of manufacturing techniques and/or tolerances, are expected. For example, a region illustrated or described as square or rectangular can have rounded or curved features, and regions shown as straight lines may have some irregularity. Thus, the regions illustrated in the figures are schematic and their shapes are not intended to illustrate the precise shape of a region of a device and are not intended to limit the scope of the disclosure. Additionally, sizes of structures or regions may be exaggerated relative to other structures or regions for illustrative purposes and, thus, are provided to illustrate the general structures of the present subject matter and may or may not be drawn to scale. Common elements between figures may be shown herein with common element numbers and may not be subsequently re-described.

The present disclosure relates generally to devices for impinging light on tissue to induce one or more biological effects and more particularly to phototherapeutic illumination devices and light guide assemblies with emission-directing structures. Emission-directing structures include arrangements of light guides and light guide positioners that may be provided with titled arrangements for directing light emissions toward target tissue. An exemplary light guide may define an optical axis therethrough, and an exemplary light guide positioner may include one or more features that are tilted with respect to the optical axis. Certain tilted features may relate to offset incisor tabs and/or angled biting surfaces that engage within the oral cavity for repeatedly orienting the optical axis in a target direction.

Aspects of the present disclosure relate to devices and methods for impinging light on a mammalian tissue, for example within a body and/or a body cavity of a patient, where the light may include at least one characteristic that exerts or induces at least one biological effect within or on the tissue. Exemplary tissues include those of the upper respiratory tract, including tissues and cavities that are accessible via the oral cavity, such as the nasopharynx and the oropharynx. Biological effects may include at least one of inactivating and inhibiting growth of one or more combinations of microorganisms and pathogens, including but not limited to viruses, bacteria, fungi, and other microbes, among others. Biological effects may also include one or more of upregulating and/or downregulating a local immune response, stimulating enzymatic generation of nitric oxide to increase endogenous stores of nitric oxide, releasing nitric oxide from endogenous stores of nitric oxide, and inducing an anti-inflammatory effect. Wavelengths of light may be selected based on at least one intended biological effect for one or more of the targeted tissues and the targeted microorganisms and/or pathogens. In certain aspects, wavelengths of light may include visible light in any number of wavelength ranges based on the intended biological effect. Further aspects involve light impingement on tissue for multiple microorganisms and/or multiple pathogenic biological effects, either with light of a single peak wavelength or a combination of light with more than one peak wavelength. Devices and methods for light treatments are disclosed that provide light doses for inducing biological effects on various targeted pathogens and targeted tissues with increased efficacy and reduced cytotoxicity. Light doses may include various combinations of irradiances, wavelengths, and exposure times, and such light doses may be administered continuously or discontinuously with a number of pulsed exposures.

Exemplary devices and methods relate to treating, preventing, and/or reducing the biological activity of pathogens while they are in one or more areas of the upper respiratory tract and hopefully before they travel to the lungs or elsewhere in the body. Devices and methods as disclosed herein may prevent or reduce infections by reducing microbial load along one or more portions of the upper respiratory tract, decreasing the ability for penetration into cells at the site of infection, and amplifying host defense systems, all of which may reduce or avoid the need for traditional antimicrobial medicines. In further aspects, devices and methods for light irradiation of tissues as disclosed herein may be provided to supplement and/or enhance the effects of traditional antimicrobial medicines.

Embodiments of the present disclosure may administer light at one or more wavelengths as a pre-exposure prophylaxis or a post-exposure prophylaxis in order to target pathogens in or on tissue of the upper respiratory tract and/or amplify host defense systems. Embodiments of the present disclosure may be used to prevent and/or treat respiratory infections and other infectious diseases. For example, in certain embodiments, a hand-held illumination device may administer light at one or more wavelengths as a prophylactic measure to counteract invading viral pathogens and corresponding diseases that may originate in the respiratory tract. In a specific example, light may be administered that reduces viral infectivity and incidence of COVID-19 in individuals who have been infected or believe they may have been exposed to SARS-CoV-2 virus. In certain aspects, illumination devices of the present disclosure may be referred to as phototherapeutic and/or phototherapy devices.

The term “phototherapy” or “phototherapeutic” generally relates to the therapeutic use of light for, by way of example, treating and/or preventing microbial infections, including viral infections of the upper respiratory tract. The mechanisms by which certain wavelengths of light are effective can vary, depending on the wavelength that is administered and the targeted microorganisms and/or pathogens. Biological effects, including antimicrobial effects, can be provided over a wide range of wavelengths, including ultraviolet (UV) ranges, visible light ranges, and infrared (IR) ranges, and combinations thereof.

Various wavelengths of visible light may be irradiated on human tissue with little or no impact on tissue viability. In certain embodiments, various wavelengths of visible light may elicit antimicrobial and/or anti-pathogenic behavior in tissue of the respiratory tract, including any of the aforementioned biological effects. For example, light with a peak wavelength in a range from 400 nanometers (nm) to 450 nm may inactivate microorganisms that are in a cell-free environment and/or inhibit replication of microorganisms that are in a cell-associated environment and/or stimulate enzymatic generation of nitric oxide, while also upregulating a local immune response in target tissue. In this regard, light with a peak wavelength in a range from 400 nm to 450 nm may be well suited for fighting invading viral pathogens and corresponding diseases that may originate in the respiratory tract, including Orthomyxoviridae (e.g., influenza), common colds, coronaviridae (e.g., coronavirus), picornavirus infections, tuberculosis, pneumonia, bronchitis, and sinusitis. In certain embodiments, red or near-infrared (NIR) light (e.g., peak wavelength range from 630 nm to 1,000 nm) may be useful to provide anti-inflammatory effects and/or to promote vasodilation. Anti-inflammatory effects may be useful in treating disorders, particularly microbial disorders that result in inflammation along the respiratory tract. In this regard, red light may be used as part of treatment protocols that reduce any tissue inflammation that may result from exposure to blue light, which may positively impact cell viability, thereby lowering cytotoxicity even further. A decrease in inflammation can be beneficial when treating viral infections, particularly when a virus can elicit a cytokine storm and/or inflammation can result in secondary bacterial infections. Accordingly, the combination of blue light, such as light at around 425 nm, and red light at one or more anti-inflammatory wavelengths, can provide a desirable combination of biological effects.

Depending on the application, other wavelength ranges of light may also be administered to human tissue. For example, UV light (e.g., UV-A light having a peak wavelength in a range of from 315 nm to 400 nm, UV-B light having a peak wavelength in a range of from 280 nm to 315 nm, and UV-C light having a peak wavelength in a range from 200 nm to 280 nm) may be effective for inactivating microorganisms that are in a cell-free environment and/or inhibit replication of microorganisms that are in a cell-associated environment and/or stimulate enzymatic generation of nitric oxide. However, overexposure to UV light may lead to cytotoxicity concerns in associated tissue. It may therefore be desirable to use shorter cycles and/or lower doses of UV light than corresponding treatments with only visible light. In certain embodiments, light with a peak wavelength in a range from 385 nm to 450 nm may be provided to elicit an antimicrobial and/or anti-pathogenic effect. In further embodiments, such wavelengths of light may be used in treatment protocols that also administer anti-inflammatory light. In certain aspects, light sources may be provided with light characteristics in the visible spectrum, for example with light emissions with peak wavelengths primarily in a range from 400 nm to 700 nm. Depending on the target application, light characteristics may also include IR or NIR peak wavelengths at or above 700 nm, or UV peak wavelengths at or below 400 nm. As used herein, light may include visual and non-visual electromagnetic radiation with single or multiple peak wavelengths in a range from 180 nm to 4,000 nm.

Doses of light to induce one or more biological effects may be administered with one or more light characteristics, including peak wavelengths, radiant flux, and irradiance to target tissues. Irradiances to target tissues may be provided in a range from 0.1 milliwatts per square centimeter (mW/cm²) to 200 mW/cm², or in a range from 5 mW/cm² to 200 mW/cm², or in a range from 5 mW/cm² to 100 mW/cm², or in a range from 5 mW/cm² to 60 mW/cm², or in a range from 60 mW/cm² to 100 mW/cm², or in a range from 100 mW/cm² to 200 mW/cm². In certain embodiments, irradiances in a range from 0.1 watts per square centimeter (W/cm²) to 10 W/cm² may be safely pulsed to target tissue. Such irradiance ranges may be administered in one or more of continuous wave and pulsed configurations, including light-emitting diode (LED)-based photonic devices that are configured with suitable power (radiant flux) to irradiate a target tissue with any of the above-described ranges.

Light sources may include one or more of LEDs, organic LEDs (OLEDs), lasers, and other lamps according to aspects of the present disclosure. Lasers may be used for irradiation in combination with optical fibers or other delivery mechanisms. LEDs are solid state electronic devices capable of emitting light when electrically activated. LEDs may be configured across many different targeted emission spectrum bands with high efficiency and relatively low costs. Accordingly, LEDs may be used as light sources in photonic devices for phototherapy applications. Light from an LED is administered using a device capable of delivering the requisite power to a targeted treatment area or tissue. High power LED-based devices can be employed to fulfill various spectral and power needs for a variety of different medical applications. LED-based photonic devices described herein may be configured with suitable power to provide irradiances as high as 100 mW/cm² or 200 mW/cm² in the desired wavelength range. An LED array in this device can be incorporated into an irradiation head, hand piece, and/or as an external unit.

In addition to various sources of light, the principles of the present disclosure are also applicable to one or more other types of directed energy sources. As used herein, a directed energy source may include any of the various light sources previously described, and/or an energy source capable of providing one or more of heat, IR heating, resistance heating, radio waves, microwaves, soundwaves, ultrasound waves, electromagnetic interference, and electromagnetic radiation that may be directed to a target body tissue. Combinations of visual and non-visual electromagnetic radiation may include peak wavelengths in a range from 180 nm to 4,000 nm. Illumination devices as disclosed herein may include a light source and another directed energy source capable of providing directed energy beyond visible and UV light. In other embodiments, the other directed energy source capable of providing directed energy beyond visible and UV light may be provided separately from illumination devices of the present disclosure.

Exemplary illumination devices as described herein may be configured to treat tissues within a variety of body cavities. For example, the devices described herein may be configured to treat, prevent, and/or reduce the biological activity of pathogens along tissues of the upper respiratory tract that are accessible via the oral cavity. Such tissues include the pharynx and/or the oropharynx, trachea, and/or esophagus. Representative types of illumination devices described herein may be suitable for positioning at or partially within a patient's mouth for delivery of light to the target tissues. As dimensions of patients' mouths and tongues can vary in scale, challenges exist in repeatably delivering light to hard-to-reach tissues, such as the oropharynx. For example, light emissions may interact with upper and lower portions of the oral cavity, including the tongue, thereby creating some shadowing effects for the oropharynx. Aspects of the present disclosure provide emission-directing structures suitable for engaging with various surfaces within the oral cavity for improving light delivery to target tissues. Such structures include light guides and associated light guide positioners that effectively angle light through the oral cavity to more directly impinge the oropharynx.

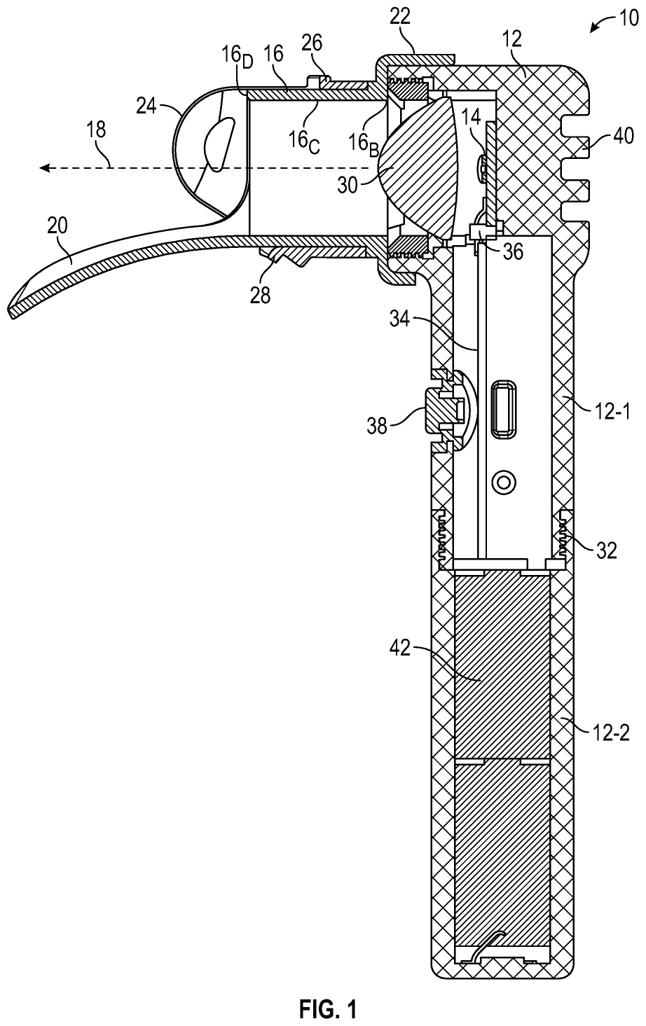

FIG. 1 is cross-section of an illumination device 10 with emission-directing structures according to the present disclosure. The illumination device 10 is configured to be a handheld device for positioning at least partially within the oral cavity of a patient (also referred to herein as a “user”). The illumination device 10 includes a housing 12 and one or more light sources 14, such as LEDs, that are arranged within the housing 12. A light guide 16 is affixed to the housing 12 such that a base end 16_{B} of the light guide 16 is proximate the one or more light sources 14, a distal end 16_{D} of the light guide 16 is positioned away from the one or more light sources 14, and a central region 16c of the light guide 16 is between the base end 16_{B} and the distal end 16_{D}. The light guide 16 defines an optical axis 18 for a direction of light propagating from the base end 16_{B} to the distal end 16_{D} and out of the light guide 16. The optical axis 18 may generally be parallel to a length of the light guide 16 defined by the walls of the light guide 16. The optical axis 18 may correspond with a center point of highest intensity and/or irradiation associated with a beam of emission from the light source 14 that is directed through the light guide 16 and toward a target surface. The light guide 16 may further include a tongue depressor 20 for displacing a user's tongue when inserted into the user's mouth. The tongue depressor 20 may be formed as an extension from the distal end 16_{D} that is curved downward from the light guide 16. In certain aspects, the tongue depressor 20 is an integral single piece with the light guide 16. The light guide 16 and tongue depressor 20 may be formed of a rigid material, such as rigid plastic materials, so that the light guide 16 and tongue depressor 20 do not deform when positioned within the oral cavity. Rather, the light guide 16 and the tongue depressor 20 may serve to displace soft tissue, including the tongue, to provide a more direct path for light to target tissue.

In certain embodiments, the light guide 16 forms a hollow core that defines a light-transmissive pathway along the optical axis 18 for propagating light from the light source 14. The hollow core may be formed by light-reflective and/or light-blocking walls of the light guide 16. An exemplary configuration includes cylindrical walls of the light guide 16 that extend between the base end 16_{B} and the distal end 16_{D}. The base end 16_{B} and the distal end 16_{D} may each form hollow ends of the light guide 16. In certain aspects, the cylindrical walls of the light guide 16 may be formed with materials that are white in color to provide light-reflective and/or light-redirecting properties. The hollow core may provide a hollow light-transmissive pathway between the base end 16_{B} and the distal end 16_{D}. By arranging the light guide 16 in this manner, increased amounts of light from the light source 14 may be exit the light guide 20 along a direct path without interacting with the walls, while wider-angled light may be reflected and/or redirected by the light guide 16 along the optical axis 18. The light guide 16 may include a flange 22 that facilitates attachment with the housing 12, such as by way of a snap-fit connection.

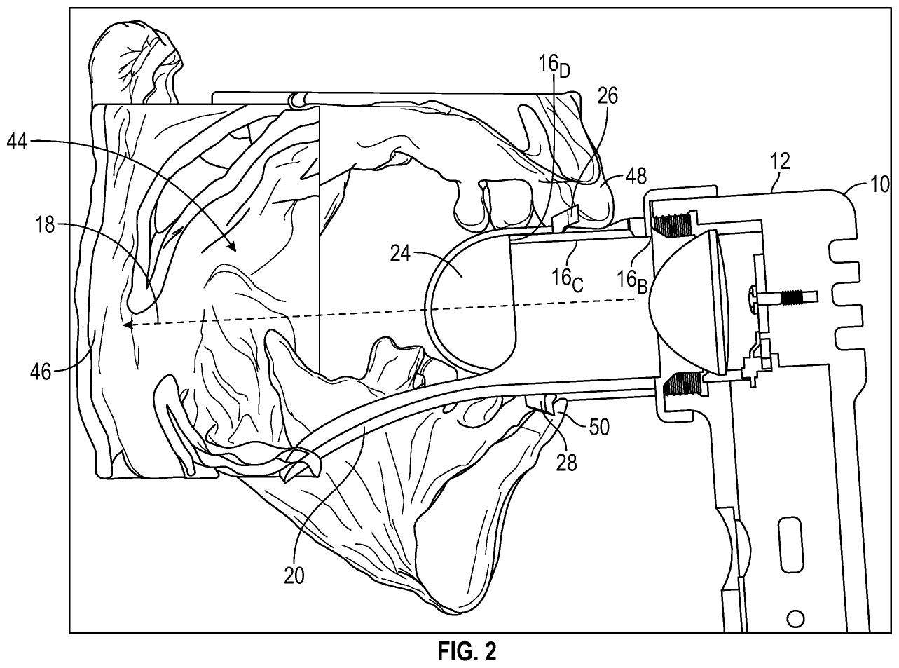

A light guide positioner 24 may be arranged about a periphery of the center region 16_{C}. In the cross-section of FIG. 1, only portions of the light guide positioner 24 are visible. As will be further illustrated in later figures, such as FIG. 2, the light guide positioner 24 may be arranged to surround the periphery of the center region 16_{C} of the light guide 16. In this regard, specific arrangements of the light guide positioner 24 may be adapted for engaging with surfaces within the oral cavity to secure the illumination device 10 and direct the optical axis 18 in a desired direction. For example, the light guide positioner 24 may include a first incisor tab 26 and a second incisor tab 28 on opposing sides of the light guide 16, such as the top side and the opposing bottom side of the light guide 16. When inserted into the oral cavity, the first incisor tab 26 may engage with back surfaces of one or more upper incisors of a patient, and the second incisor tab 28 may engage with back surfaces of one or more lower incisors of the patient.

Accordingly, the light guide positioner 24 and incisor tabs 26, 28 may secure portions of the light guide 16 at an intended position for phototherapy. In certain embodiments, the first incisor tab 26 is arranged closer to the base end 16_{B} of the light guide 16 than the second incisor tab 28. In this manner, when the first and second incisor tabs 26, 28 are respectively engaged with upper and lower incisors of a patient, the optical axis 18 will be tilted downward relative to the oral cavity in a direction that targets the various portions of the pharynx, including the oropharynx. Additionally, the tongue depressor 20 may also be tilted downward to apply increased pressure on the user's tongue to further expose the oropharynx for receiving light. Notably, such a configuration may provide increased irradiance to the oropharynx when the lower jaw of a patient is opened to receive light guide positioner 24. In certain embodiments, the light guide positioner 24 may comprise a material, such as silicone, that is less rigid than the light guide 16 for improved comfort within the oral cavity.

The illumination device 10 may include a lens 30 positioned between the base end 16_{B} and the one or more light sources 14. The lens 30 may be useful for controlling and directing an emission pattern of the one or more light sources 14 into the light guide 16. In certain embodiments, a portion of the lens 30, such as an apex of the lens 30, may at least partially reside within the light guide 16 to further enhance emissions along the optical axis 18. In still further embodiments, the apex of the lens 30 may be aligned with the optical axis 18 of the light guide 16.

In certain embodiments, the housing 12 may include an upper housing 12-1 and a lower housing 12-2. In certain embodiments, the upper housing 12-1 may be mechanically coupled and decoupled to the lower housing 12-2 in a tool-less manner, such as a threaded connection 32 or the like. In other embodiments, the upper housing 12-1 and the lower housing 12-2 as used herein may generally refer to upper and lower portions of a unitary single housing 12. The upper housing 12-1 may include the one or more light sources 14 and the lens 30, and the upper housing 12-1 may be mechanically coupled with the light guide 16, for example by way of the flange 22. Driver circuitry 34 for driving and/or controlling output of the one or more light sources 14 may be arranged within a portion of the upper housing 12-1 and electrically coupled with the one or more light sources 14 by way of a connector 36. A user interface element 38, such as a tactile element, button, or switch, may be positioned along the upper housing 12-1 so that a user may initiate operation of the illumination device 10. A portion of the upper housing 12-1 may include various features 40, such as trenches and/or protrusions, that increase a surface area of the housing 12 proximate the light sources 14 for heat dissipation purposes. An energy storage device 42, such as a rechargeable battery, may be positioned within the lower housing 12-2.

FIG. 2 is a partial cross-section illustrating placement of the illumination device 10 of FIG. 1 partially within an oral cavity 44 of a user or patient. For illustrative purposes, only a portion of tissue that includes the oropharynx 46 is illustrated within a superimposed box relative to upper and lower jaws and teeth. As illustrated, when the lower jaw is opened to receive the illumination device 10, the lower jaw is angled downward relative to the upper jaw. According to aspects of the present disclosure, one or more elements of the light guide positioner 24 are tilted relative to the optical axis 18 and the light guide 16 to compensate for the open jaw position and direct the optical axis 18 toward the oropharynx 46. As illustrated, the first incisor tab 26 may engage with back surfaces of one or more upper incisors 48, and the second incisor tab 28 may engage with back surfaces of one or more lower incisors 50. Accordingly, the light guide positioner 24 and incisor tabs 26, 28 may effectively index the distal end 16_{D} of the light guide 16 past the upper and lower incisors 48, 50 so that the optical axis 18 is directed toward target tissue. When the first incisor tab 26 is arranged closer to the base end 16_{B} of the light guide 16 than the second incisor tab 28 as described above, the optical axis 18 may be tilted downward in a direction towards the oropharynx 46. Such tilting may also serve to position the tongue depressor 20 to further depress the user's tongue and provide increased access to the oropharynx 46.

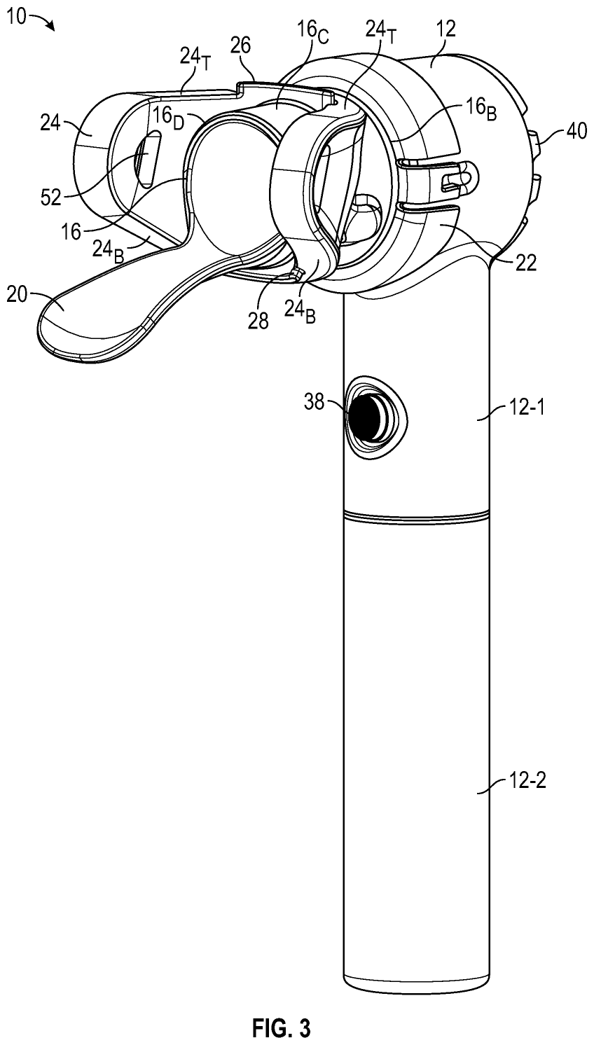

FIG. 3 is a front perspective view of the illumination device 10 of FIG. 1. As illustrated, the light guide positioner 24 is arranged about a periphery of the light guide 16 and in particular about a periphery of the center region 16C of the light guide 16. The light guide positioner 24 may embody a mouthpiece shape such that a top surface 24_{T} and a bottom surface 24_{B} thereof respectively form an upper biting surface and a lower biting surface for teeth when positioned as illustrated in FIG. 2. For example, portions of the top surface 24_{T} and the bottom surface 24_{B} that are closest to the incisor tabs 26, 28 may form biting surfaces for anterior teeth while portions of the top surface 24_{T} and the bottom surface 24_{B} that are farthest from the incisor tabs 26, 28 may form bite surfaces for posterior teeth. In this regard, the light guide positioner 24 may also be referred to as a mouthpiece in such embodiments. In certain arrangements, one or more holes 52 may be formed through portions of the light guide positioner 24 to allow air flow for easier breathing during use.

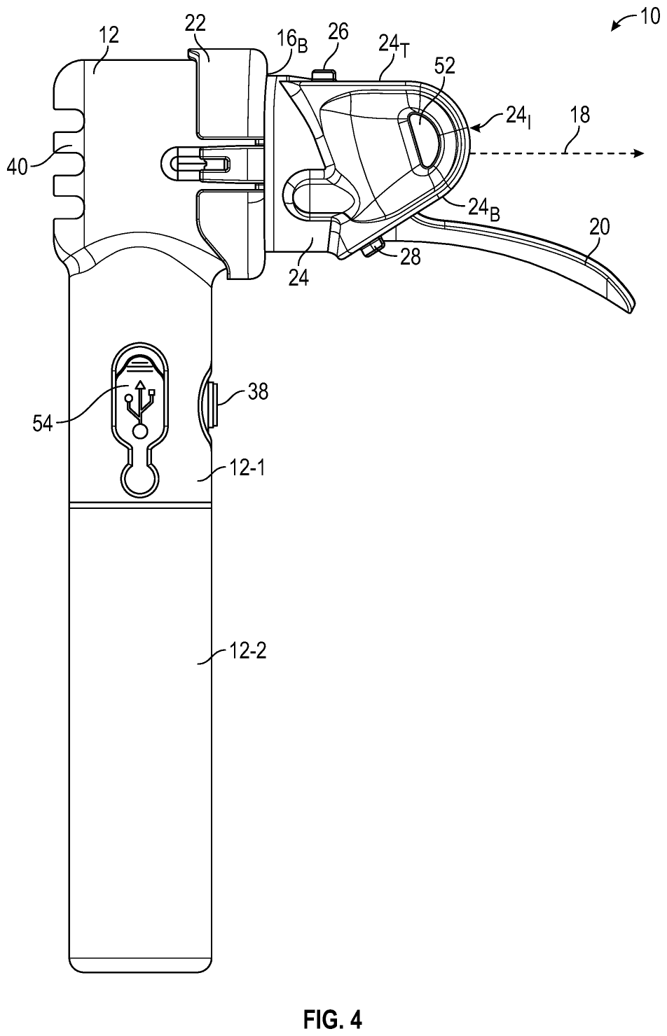

FIG. 4 is a side view of the illumination device 10 of FIG. 3. As illustrated in this view, the illumination device 10 may include a port 54 for one or more of charging the illumination device 10, providing data for storing within the illumination device 10, and accessing data stored to illumination device 10. From the side view, the orientation of the light guide positioner 24 relative to optical axis 18 is more readily visible. In particular, at least a portion of the bottom surface 24_{B} is angled upward toward the optical axis 18 while at least a planar portion of the top surface 24_{T} is parallel or closer to parallel with the optical axis 18 than the bottom surface 24_{B}. In this manner, an inflection point 24_{I} between the top surface 24_{T} and the bottom surface 24_{B} of the light guide 16 may be positioned offset from the optical axis 18. In particular, the inflection point 24_{I} may be arranged above the optical axis 18 such that the optical axis 18 is directed between the inflection point 24_{I} and the tongue depressor 20.

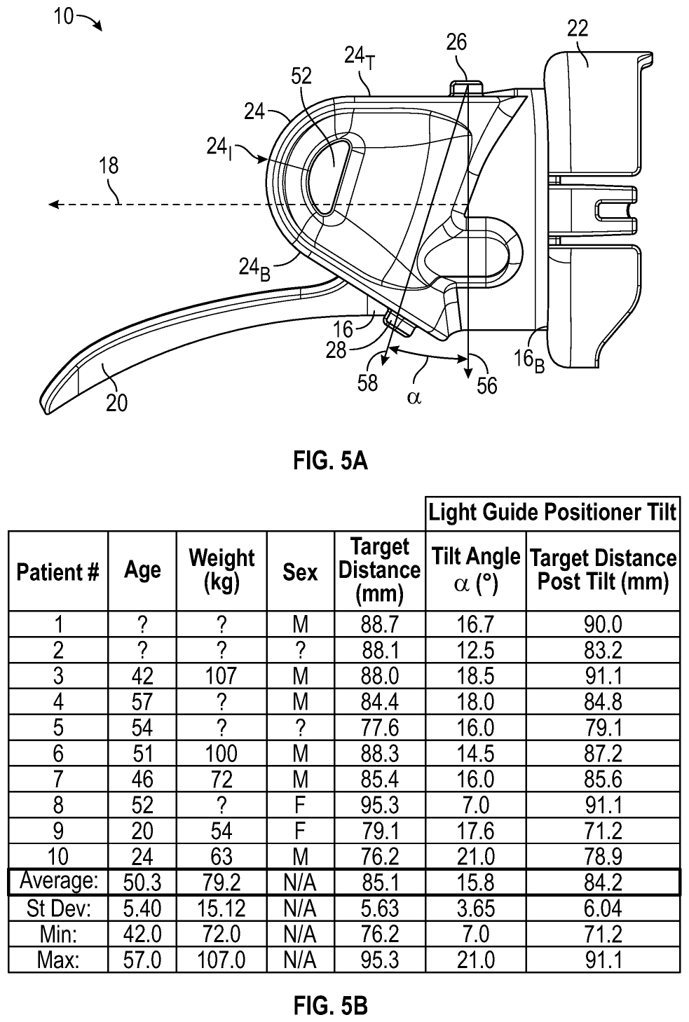

FIG. 5A is a side view of a portion of the illumination device 10 of FIG. 3 that illustrates the light guide positioner 24 arranged about the light guide 16. As described herein, the arrangement of the light guide positioner 24 and the light guide 16 may collectively be referred to as a light guide assembly. For illustrative purposes, the remainder of the illumination device 10 is omitted. The illumination device 10 is configured with a tilted relationship between the light guide positioner 24 and the light guide 16 for repeatably directing the optical axis 18 toward a patient's oropharynx during use. The tilted relationship may be defined as an angle of rotation, or tilt angle α, for various elements of the light guide positioner 24 relative to the optical axis 18. For example, since the first incisor tab 26 on the top side of the light guide 16 may be arranged closer to the base end 16_{B} than the second incisor tab 28 on the bottom side of the light guide 16, the tilt angle α may be defined as follows. In FIG. 5A, a first line 56 may be defined perpendicular to the optical axis 18 and/or or parallel with a plane of the based end 16_{B}, and a second line 58 may be defined that intersects the first incisor tab 26 and the second incisor tab 28. In this manner, the tilt angle α is defined as an angle between the first line 56 and the second line 58. The tilt angle α may also correspond with an angle of rotation between the inflection point 24_{I} and the optical axis 18. Accordingly, the tilt angle α refers to an orientation of the light guide positioner 24 that is offset from the optical axis 18 so that when engaged within the oral cavity, the optical axis 18 may effectively be pointed downward according to the tile angle α for increasing irradiation to the oropharynx.

In order to determine a suitable tile angle α, or ranges thereof, that may generally be applicable for a majority of the human population, a study of various jaw anatomies was performed. FIG. 5B is a table listing various parameters for the study and simulated tilt angle α values. As an initial step, magnetic resonance imaging (MRI) scans were obtained for ten patients of various ages, weights, and genders. The ten patients were chosen from relatively clear MRI scans available from the Cancer Imaging Archive. A target distance is defined as a distance in millimeters (mm) between the upper incisor tip to the posterior pharyngeal wall at the oropharynx. The collected MRI scans were converted to full solid models to study the anatomical behavior when the lower jaw is opened relative to the upper jaw to accommodate the illumination devices of the present disclosure. The suitable tilt angle α for directing the optical axis toward the oropharynx as described for FIG. 5A was determined for each of the patients and is summarized in FIG. 5B along with slight adjustments to the target distance based on the open jaw. Tilt angles α ranged from as high as 21 degrees to as low as 7 degrees, with an average of about 16 degrees. In certain embodiments, the tilt angle α for the illumination device 10 of FIG. 5A may be provided in a range from 8 degrees to 22 degrees, which is roughly two standard deviations from the average. In still further embodiments, the tilt angle α may include ranges with lower values, such as a range from 5 degrees to 22 degrees, or in a range from 5 degrees to 20 degrees, or about 6 degrees with a tolerance of plus or minus 1 degree. In some instances, tilt angles α with lower values, for example as low as 5 or 6 degrees with a tolerance of plus or minus 1 degree, may apply reduced pressure on a user's tongue while still displacing the tongue sufficiently to provide a light path to the oropharynx. In certain embodiments, the target distance as defined above may be set in a range from 70 mm to 96 mm.

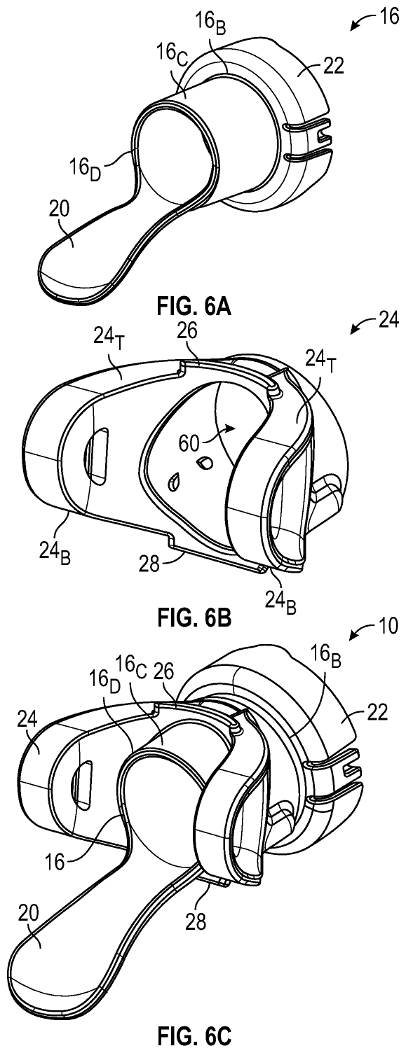

FIGS. 6A to 6C illustrate perspective views of the light guide 16, the light guide positioner 24, and a light guide assembly that includes both the light guide 16 and the light guide positioner 24. FIG. 6A is a perspective view of the light guide 16 of FIG. 5A illustrating the base end 16_{B}, the distal end 16_{D}, and the central region 16c. A hollow light-transmissive pathway may be defined by interior walls of the light guide 16 such that light may exit in a direction proximate the tongue depressor 20. FIG. 6B is a perspective view of the light guide positioner 24 of FIG. 5A. As illustrated, a central opening 60 may be formed within the light guide positioner 24 for receiving the central region 16c of the light guide 16 of FIG. 6A. FIG. 6C illustrates a perspective view of the fully assembled light guide assembly that includes both the light guide 16 of FIG. 6A and the light guide positioner 24 of FIG. 6B. In certain embodiments, the light guide 16 and the light guide positioner 24 may be removable structures that are securely joined to one another. In other embodiments, the light guide positioner 24 may be molded to the light guide 16 to form a single unitary structure that is not readily disassembled.

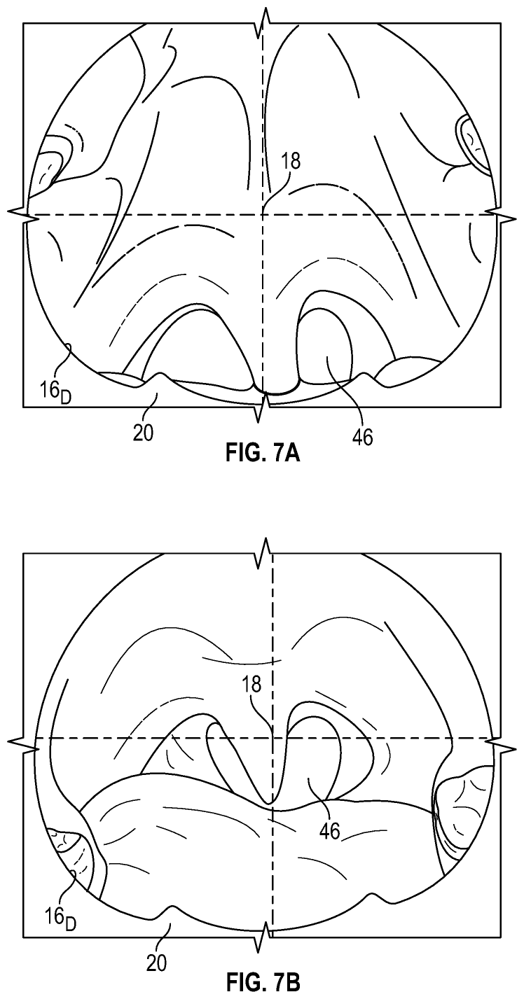

FIGS. 7A and 7B are illustrations of images comparing the optical axis 18 in a patient's oral cavity for illumination devices with and without tilted arrangements as described above. For both images the illustrations are based on, a camera was positioned within the light guide in a same direction as the optical axis 18. In each image, portions of the distal end 16_{D} and the tongue depressor 20 are visible. FIG. 7A is an illustration of an image with an illumination device that is not arranged with a tilt between the light guide positioner and the light guide as described above. When inserted into the patient's mouth, the optical axis 18, which may correspond with a highest intensity of light in the emission pattern, is directed toward a roof of the patient's mount. While suitable light that is off axis from the optical axis 18 may still reach the patient's oropharynx 46, some light loss is realized. In contrast, FIG. 7B is an illustration of an image with the illumination device 10 according to FIGS. 1-6C that includes the above-described titled arrangement in the same patient's mouth. As shown, the optical axis 18 is now pointed directly at the oropharynx 46 with only the patient's uvula therebetween. Accordingly, increased amounts of light from the illumination device 10 may reach the oropharynx 46 for treatment. It is also contemplated that the patient's head tilt may also adjust amounts of light that reach the oropharynx 46. For example, when the patient's head is tilted downward such that the patient's chin is closer to the patient's chest, the titled arrangement of the light guide positioner and the corresponding angle of the tongue depressor 20 may increase an opening within the oral cavity for light to reach the oropharynx 46.

It is contemplated that any of the foregoing aspects, and/or various separate aspects and features as described herein, may be combined for additional advantage. Any of the various embodiments as disclosed herein may be combined with one or more other disclosed embodiments unless indicated to the contrary herein.

Those skilled in the art will recognize improvements and modifications to the preferred embodiments of the present disclosure. All such improvements and modifications are considered within the scope of the concepts disclosed herein and the claims that follow.

What is claimed is:

1. A light guide assembly comprising:
a light guide configured to be optically coupled to at least one light source, the light guide comprising a base end that is proximate the at least one light source, a distal end, and a center region that is between the base end and the distal end; and
a light guide positioner arranged about a periphery of the center region, the light guide positioner comprising a first incisor tab and a second incisor tab that are positioned on opposing sides of the center region, the first incisor tab being positioned closer to the base end of the light guide than the second incisor tab.

2. The light guide assembly of claim 1, wherein the first incisor tab is arranged to engage with a back surface of an upper incisor and the second incisor tab is arranged to engage with a back surface of a lower incisor to position a portion of the light guide within an oral cavity of a patient.

3. The light guide assembly of claim 1, wherein:
the light guide defines an optical axis in a direction from the base end to the distal end;
the light guide positioner comprises an upper biting surface and a lower biting surface; and
the lower biting surface is angled toward the optical axis such that at least a portion of the upper biting surface is closer to parallel with the optical axis than the lower biting surface.

4. The light guide assembly of claim 1, wherein the light guide defines an optical axis in a direction from the base end to the distal end, and a tilt angle is formed between a first line perpendicular to the optical axis and a second line that intersects the first incisor tab and the second incisor tab, the tilt angle being in a range from 5 to 22 degrees.

5. The light guide assembly of claim 4, wherein the tilt angle is in a range from 5 to 20 degrees.

6. The light guide assembly of claim 1, further comprising a tongue depressor that extends from the distal end of the light guide.

7. An illumination device comprising:
at least one light source;
driver circuitry configured to drive the at least one light source;
a light guide optically coupled to the at least one light source, the light guide comprising a base end that is proximate the at least one light source, a distal end, and a center region that is between the base end and the distal end; and
a light guide positioner arranged about a periphery of the center region, the light guide positioner comprising a first incisor tab and a second incisor tab that are positioned on opposing sides of the center region, the first incisor tab being positioned closer to the base end of the light guide than the second incisor tab.

8. The illumination device of claim 7, wherein the first incisor tab is arranged to engage with a back surface of an upper incisor and the second incisor tab is arranged to engage with a back surface of a lower incisor to position a portion of the light guide within an oral cavity of a patient.

9. The illumination device of claim 7, wherein:
the light guide defines an optical axis in a direction from the base end to the distal end;
the light guide positioner comprises an upper biting surface and a lower biting surface; and
the lower biting surface is angled toward the optical axis such that at least a portion of the upper biting surface is closer to parallel with the optical axis than the lower biting surface.

10. The illumination device of claim 7, wherein the light guide defines an optical axis in a direction from the base end to the distal end, and a tilt angle is formed between a first line perpendicular to the optical axis and a second line that intersects the first incisor tab and the second incisor tab, the tilt angle being in a range from 5 to 22 degrees.

11. The illumination device of claim 10, wherein the tilt angle is 6 degrees with a tolerance of plus or minus 1 degree.

12. The illumination device of claim 7, further comprising a tongue depressor that extends from the distal end of the light guide.

13. The illumination device of claim 7, further comprising a lens positioned between the at least one light source and the light guide, wherein an apex of the lens is positioned within the light guide.

14. An illumination device comprising:
at least one light source;
driver circuitry configured to drive the at least one light source;
a light guide optically coupled to the at least one light source, the light guide comprising a base end that is proximate the at least one light source, a distal end, and a center region that is between the base end and the distal end, the light guide forming an optical axis in a direction from the base end to the distal end; and
a light guide positioner arranged about a periphery of the center region, the light guide positioner comprising an upper biting surface, a lower biting surface, and an inflection point defined between the upper biting surface and the lower biting surface, the inflection point being offset from the optical axis.

15. The illumination device of claim 14, further comprising a tongue depressor that extends from the distal end of the light guide.

16. The illumination device of claim 15, wherein the optical axis is positioned between the inflection point of the light guide positioner and the tongue depressor.

17. The illumination device of claim 14, wherein the light guide positioner comprises a first incisor tab proximate the upper biting surface and a second incisor tab proximate the lower biting surface, and the first incisor tab is positioned closer to the base end of the light guide than the second incisor tab.

18. The illumination device of claim 17, wherein a tilt angle is formed between a first line perpendicular to the optical axis and a second line that intersects the first incisor tab and the second incisor tab, the tilt angle being in a range from 5 to 22 degrees.

19. The illumination device of claim 18, wherein the tilt angle is in a range from 5 to 20 degrees.

20. The illumination device of claim 14, wherein the lower biting surface is angled toward the optical axis such that at least a planar portion of the upper biting surface is closer to parallel with the optical axis than the lower biting surface.
